(19) United States
(12) Patent Application Publication
(10) Pub. No.: US 2005/0192659 A1
(43) Pub. Date: Sep. 1, 2005

(54) Apparatus and method for performing a surgical procedure

(76) Inventors: Terry Dahl, Santa Barbara, CA (US); Hugh H. Trout III, Bethesda, MD (US); Stan Needle, Louisville, CO (US)

Correspondence Address: COLLIER SHANNON SCOTT, PLLC, 3050 K STREET, NW, SUITE 400, WASHINGTON, DC, 20007, US

(21) Appl. No.: 11/038,408

(22) Filed: Jan. 21, 2005

Related U.S. Application Data
(60) Provisional application No. 60/538,242, filed on Jan. 23, 2004.

(51) Int. Cl. A61F 2/06

(52) U.S. Cl. 623/1.11

(57) ABSTRACT

The present invention depicts a method and apparatus for treating an aneurysm. The method of treating an aneurysm is to first, insert a flared stent having a lumen into an artery with the flared portion extending into the lumen of the aneurysm, second, insert a bifurcation graft through the lumen of the flared stent and attach the bifurcation graft to the artery; and finally, insert an inner stent into the lumen of the bifurcation graft.

### CROSS-REFERENCE TO RELATED APPLICATIONS

The present invention relates to, and is entitled to the benefit of the earlier filing date and priority of, Application No. 60/538,242 filed on Jan. 23, 2004.

### FIELD OF THE INVENTION

The present invention relates generally an apparatus and method for use in surgical repair, more particularly for use in the repair of aneurysms.

### BACKGROUND

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm as no statistical benefit exists to do so.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is extremely high, there is also considerable mortality and morbidity associated with open surgical intervention to repair an aortic aneurysm. This intervention involves penetrating the abdominal wall to the location of the aneurysm to reinforce or replace the diseased section of the abdominal wall (i.e., abdominal aorta). A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, methods have been developed to attempt to treat an abdominal aortic aneurysm without the attendant risks of intra-abdominal surgical intervention. Although techniques have been developed that may reduce the stress, morbidity, and risk of mortality associated with surgical intervention to repair aortic aneurysms, none of the prior art systems that have been developed effectively treat the aneurysm and exclude the affected section of aorta from the pressures and stresses associated with circulation. None of the devices disclosed in the references provide a reliable and quick means to reinforce an aneurysmal artery. In addition, all of the prior references require a sufficiently large section of healthy aorta abutting the aneurysm to ensure attachment of the graft. The proximal aortic neck (i.e., above the aneurysm) is usually sufficient to support a graft's attachment means. However, when an aneurysm is located near the iliac arteries, there may be an ill-defined neck or no neck below the aneurysm. Such an ill-defined neck would have an insufficient amount of healthy aortic tissue to which to successfully attach a graft. Furthermore, much of the abdominal aortic wall may be calcified making it extremely difficult to attach a graft thereto.

Additional advantages of various embodiments of the invention are set forth, in part, in the description that follows and, in part, will be apparent to those of ordinary skill in the art from the description and/or from the practice of the invention.

### SUMMARY

The present invention is directed to a method and apparatus for treating aneurysms. The method of one embodiment of the present invention is to insert a flared stent having a lumen into an artery with the flared portion extending into the lumen of the aneurysm, second, insert a bifurcation graft through the lumen of the flared stent and attach the bifurcation graft to the artery; and finally, insert an inner stent into the lumen of the bifurcation graft.

Further embodiments of the method of using the present invention include using a repair catheter to ablate a stricture in an artery. One embodiment uses an expandable device to loosen material of the stricture and to capture the loosened material using an occlusive device placed into the artery.

An embodiment of the apparatus of the present invention is a bifurcation graft having a main body having a first leg having two legs, one of the two legs is inserted into a flared stent, and an inner stent is inserted into the same leg.

It is one object of the present invention to treat abdominal aortic aneurysms using a bifurcation graft that is inserted into two adjacent iliac arteries.

Additional advantages of the invention will be set forth in part in the description that follows, and in part will be obvious from the description, or may be learned by practice of the invention. The advantages of the invention will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments of the invention and together with the description, serve to explain the principles of the invention. Where appropriate, the same reference numerals refer to the same or similar elements.

FIG. 1 is a schematic view of an abdominal aortic aneurysm.

FIG. 2 is depicts a method of inserting an unsupported graft in an abdominal aortic aneurysm.

FIGS. 3 and 8 are schematic views of a stenosis at the orifice of the right common iliac artery.

FIG. 4 is a schematic view of a stenosis causing a stricture of the right limb of a bifurcated graft.

FIGS. 5, 6, and 7 are schematic views of possible complications while attempting to dilate a stricture using a stent.

FIG. 9 is a schematic view of a catheter with an expandable repair device according to an embodiment of the present invention.

FIG. 10 is a schematic view of a catheter with an expandable repair device in an artery with an aneurysm according to an embodiment of the present invention.

FIG. 11 is an enlarged schematic view of an embodiment of the catheter with an expandable repair device according to an embodiment of the present invention.

FIGS. 12 through 19 are schematic views of embodiments of a repair stent according to an embodiment of the present invention.

FIG. 20 is a schematic view of an abdominal aortic aneurysm with flared stents that are asymmetric according to an embodiment of the present invention.

FIG. 21 is a schematic view of the right and left limbs of a prosthetic bifurcated graft passing through an abdominal aortic aneurysm with flared stents according to an embodiment of the present invention.

FIGS. 22, 23, and 24 are schematic views of various methods to secure the limbs of the prosthetic graft to arteries with flared stents according to an embodiment of the present invention.

FIG. 25 is an exploded view of a right common iliac artery with an iliac asymmetrical stent according to an embodiment of the present invention.

### DETAILED DESCRIPTION OF EMBODIMENTS OF THE INVENTION

Reference now will be made in detail to the apparatus and methods consistent with implementations of the present invention, examples of which are illustrated in the accompanying drawings. The appended claims define the scope of the invention, and the following description does not limit that scope.

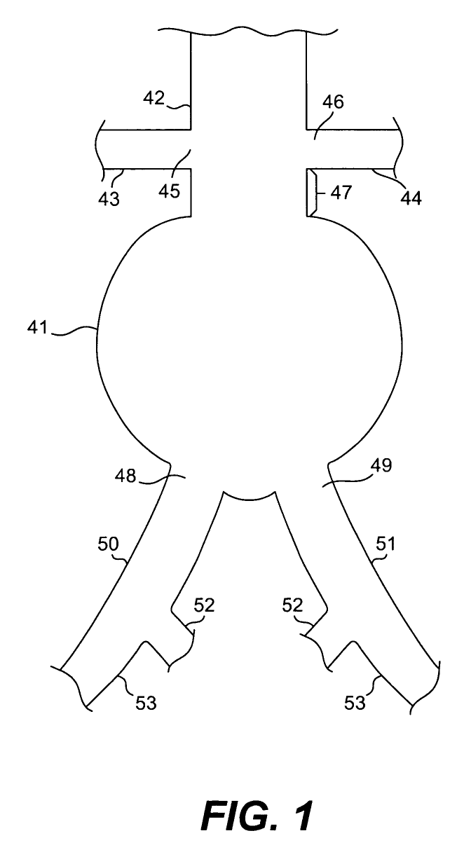

FIG. 1 depicts an abdominal aortic aneurysm (AAA) 41. In this instance, it arises from the abdominal portion of the aorta 42 and is infrarenal, that is, inferior to the right 43 and left 44 renal arteries. Typically, as is shown, there is a portion of the aorta just below the renal artery orifices 45 & 46 that is normal diameter 47. This segment 47 is often referred to as the “aortic neck”. The orifices 48 & 49 of the right common 50 and left common 51 iliac arteries arise from the distal or inferior portion of the aneurysm 41. These common iliac arteries subsequently bifurcate into the internal 52 and external 53 iliac arteries that supply arterial blood flow to the pelvis and lower extremities respectively. The following drawings and description illustrate the use of the present invention with the repair of abdominal aortic aneurysms. However, it will be obvious to those skilled in the art that the present invention could be used in the repair of other arteries or vessels or any other suitable purpose.

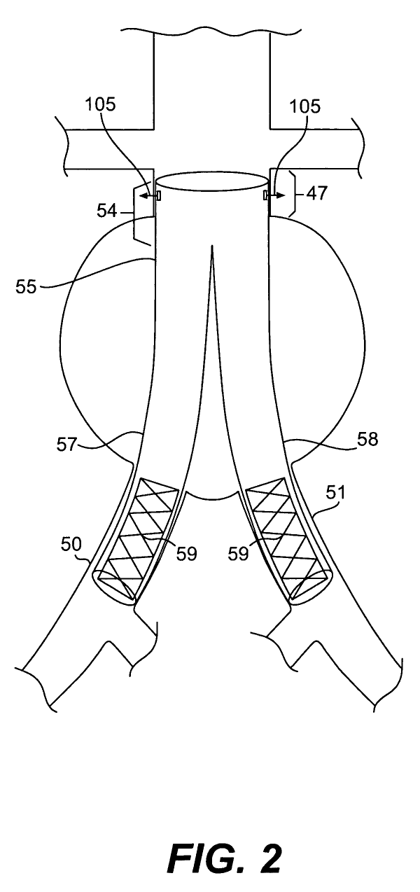

A method of inserting an unsupported graft made of fabric such as, but not limited to, polyester via an endovascular approach as treatment for an infrarenal abdominal aortic aneurysm is illustrated in FIG. 2. In this depiction, the tube portion 54 of a prosthetic bifurcation graft 55 is attached circumferentially to the full thickness of the aortic neck 47 wall with at least one surgical fastener 105. The right 57 and left 58 limbs of the bifurcation graft are attached respectively to the right 50 and left 51 common iliac arteries with stents 59.

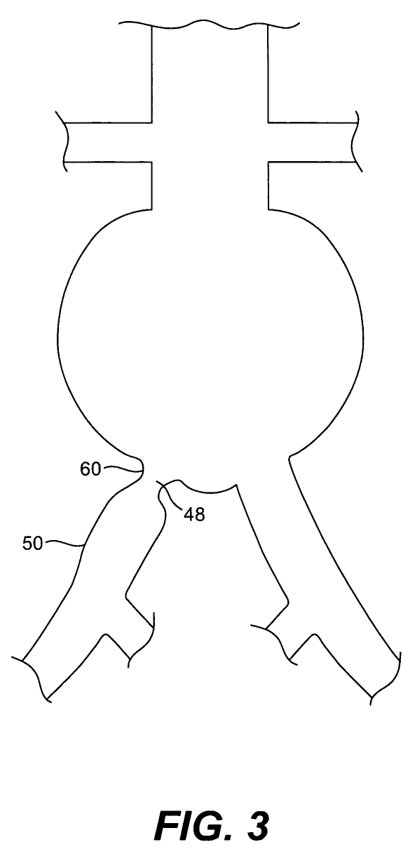

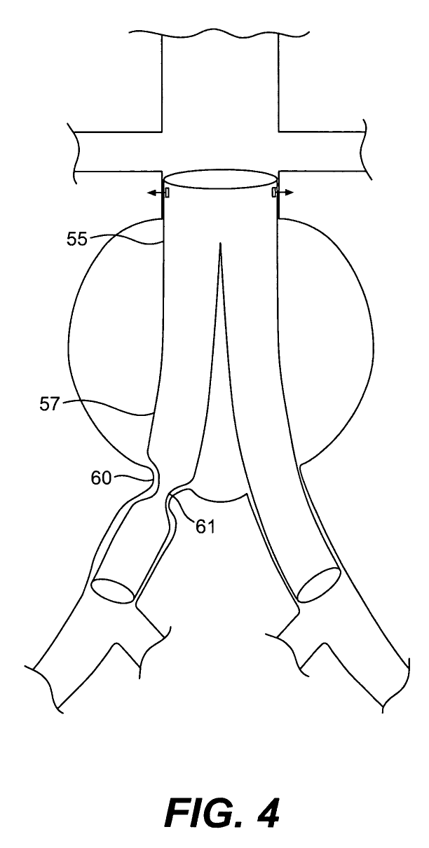

Occasionally one or both orifices of the common iliac arteries are narrowed (stenotic). FIG. 3 depicts such a stenosis 60 at the orifice 48 of the right common iliac artery 50. FIG. 4 shows such a stenosis 60 causing a stricture 61 of the right limb 57 of a bifurcation graft 55.

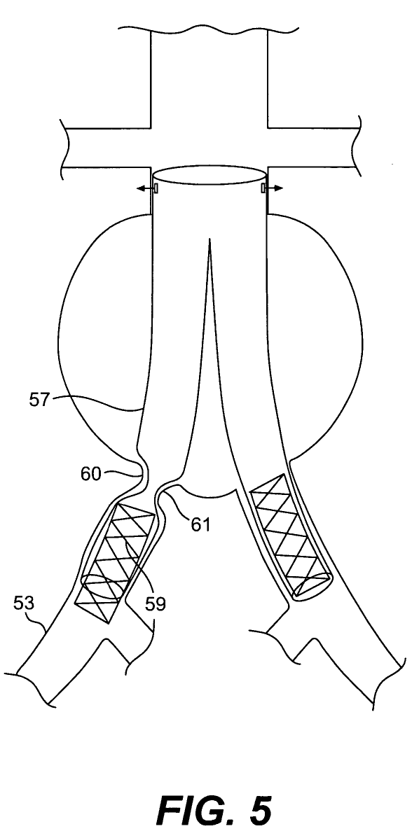

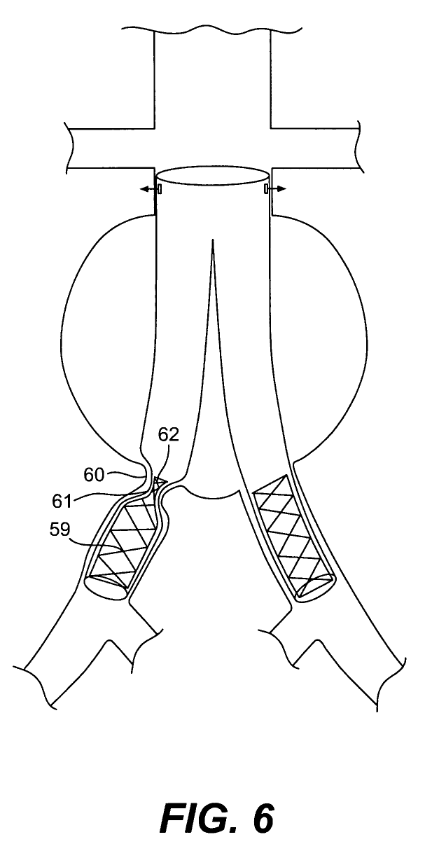

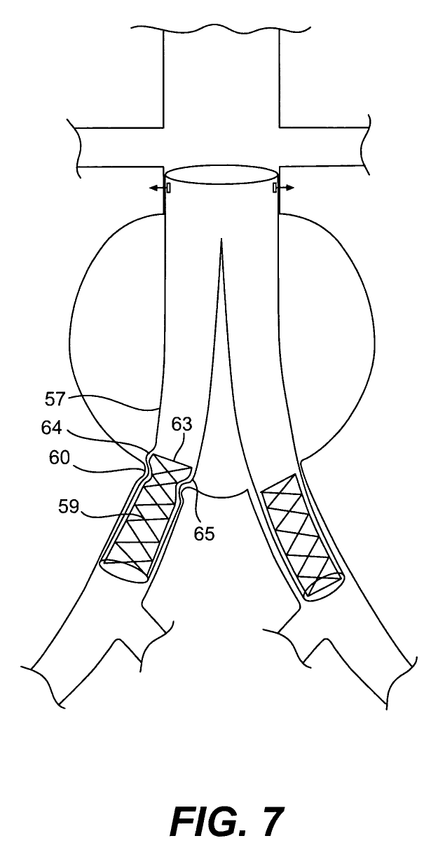

In many instances, it may be possible for the stent used to attach the graft limb to dilate this stricture without subsequent consequence. On other occasions, however, complications may occur. Three of a number of possible complications are depicted in FIGS. 5-7. FIG. 5 depicts a stent 59 that has either been misplaced or has, as a result of the rigid stricture 60, migrated distally toward the external iliac artery 53 leaving the graft limb 57 still strictured 61 at the area of the orificial stricture 60. FIG. 6 depicts a stent 59 that is correctly placed but the portion 62 of the stent 59 at the stricture 60 is unable to overcome the stricture 60 and is thus ineffective in relieving the graft stricture 61. FIG. 7 depicts a stent that is successful at dilating the stricture 60 but the stent is placed such that the proximal portion 63 of the stent 59 projects into the portion of the right graft limb 57 that is within the aneurysm. In this setting, it is possible that the portion of the graft limb that is adjacent to the proximal portion 63 of the stent 59 might fray at 64 or 65 in response to friction between the rigid stent 59 and the flexible prosthetic fabric graft limb 57.

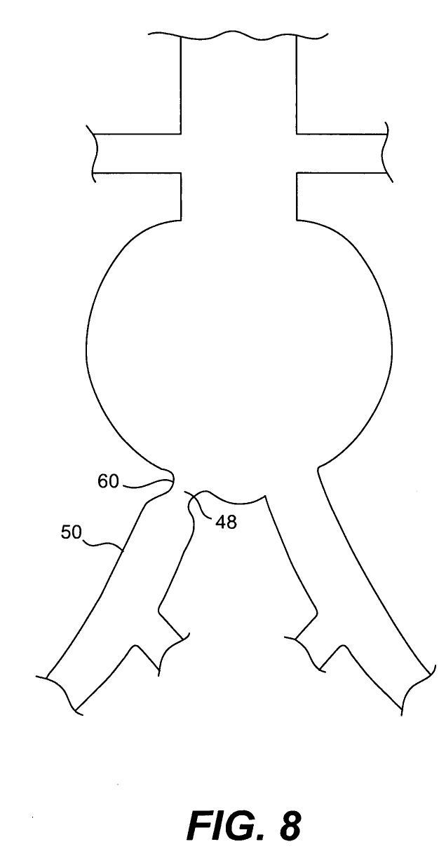

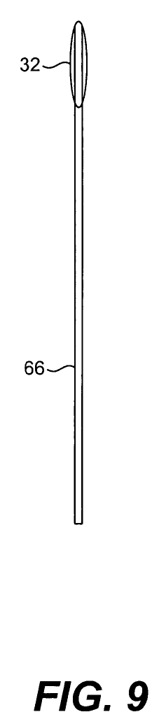

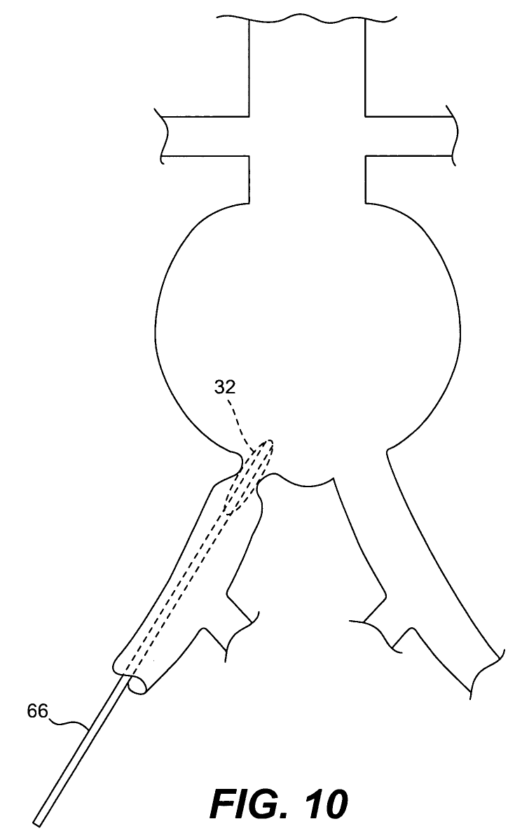

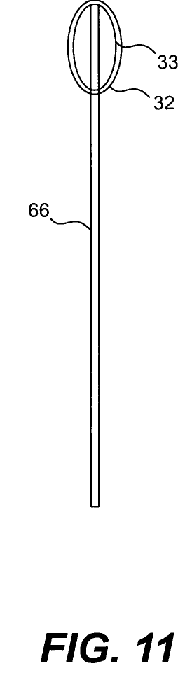

Anatomical settings may present an obstacle to maintaining long-term patency of an endovascularly placed endograft as treatment for an abdominal aortic aneurysm. As shown in FIG. 8, the orifice 48 of the common iliac artery 50 shows a typical stricture 60, most commonly caused by an atherosclerotic plaque. On occasion, it may be perceived an advantage to abolish or reduce the degree of this stricture. One way to accomplish this would be to place an ultrasound probe via a distal artery, such as the femoral artery, and fracture the plaque by intravascular ultrasound. A method according to an embodiment of the present invention, shown in FIG. 9, is to insert a repair catheter 66 into a distal artery, such as the femoral artery. At some point along the repair catheter 66 would be placed a low profile expandable device 32 made of plastic or metal or any other suitable material. Once the expandable device 32 was within the strictured artery, as shown in FIG. 10, the expandable device 32 is expanded and the plaque 4 may be loosened by vibration (piezoelectric or mechanical) or ultrasound or by any other suitable method. FIG. 11 shows an alternative embodiment in that the expandable device 32 is expanded by a balloon 33. Alternatively, the repair catheter could use any suitable method other than an expandable device to ablate the stricture.

The indications for such an approach could be the example demonstrated in an attempt to ablate or reduce an orificial stricture at the common iliac artery. Another example could be reducing the amount of plaque in the common and internal carotid arteries in someone at risk for a stroke. In this setting, loose plaque material could be captured by an occlusive or a cerebral protection (usually a filter type) device placed distally in the internal carotid artery.

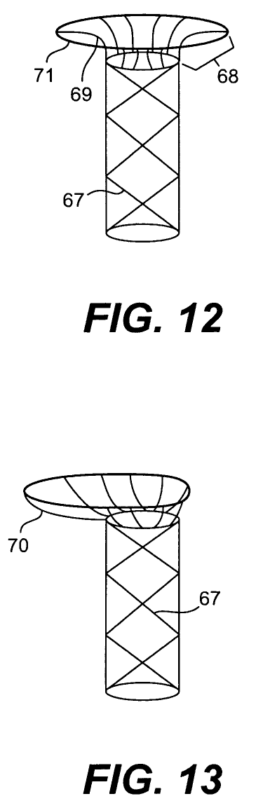

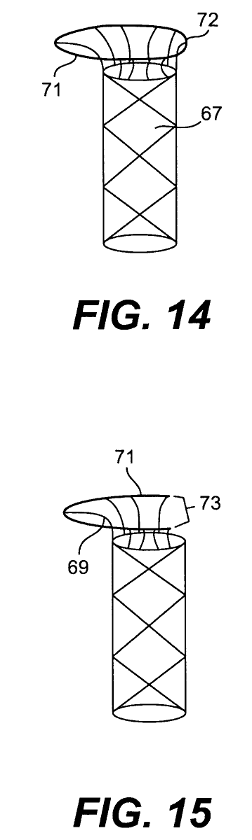

Another approach according to an embodiment of the present invention for treating a stricture or an occlusion in the common iliac artery (see FIG. 14) prior to inserting an endograft as treatment for AAA while, at the same time, reducing the likelihood of subsequent fraying of the prosthetic graft limb (see FIG. 7) is to insert a specialized stent 67 that dilates an artery along with its orifice. Examples of such stent designs according to embodiments of the present invention are shown in FIGS. 12-19.

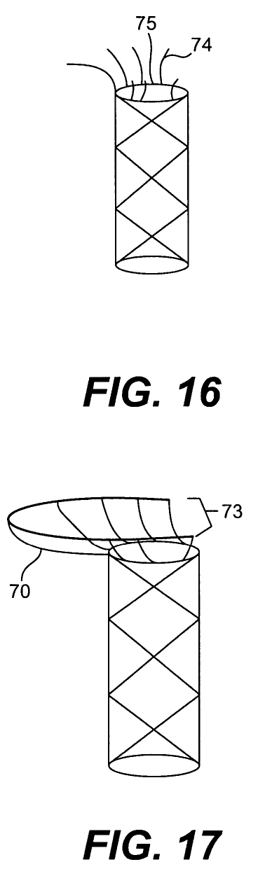

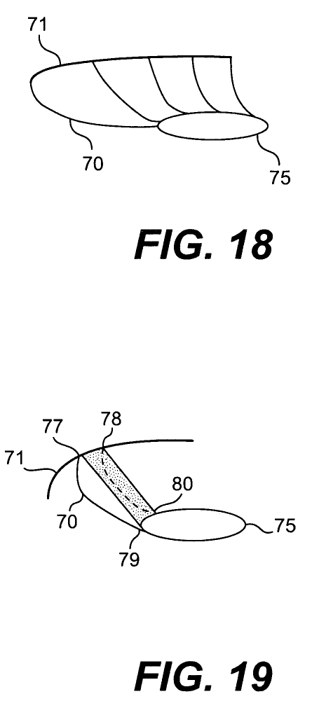

FIG. 12 depicts a balloon or self-expanding stent 67 that has one or both ends with flared portions 68. Support struts 69 connecting flared portion 68 to the main body of stent 67 can be convex 69 or concave 70 as seen in FIG. 13. The flared portion 68 can be asymmetrical 72 such as depicted in FIG. 14, a portion 73 can be omitted as shown in FIG. 15 or the outer ring 71 seen in FIG. 15 can be omitted as seen in FIG. 16 simply leaving struts (symmetric or asymmetric) 74 simply attached to inner ring 75. FIG. 17 shows missing portion 73 of flared stent 67 and is analogous to stent 67 depicted in FIG. 15 except for concave struts 70 as opposed to convex struts 69. All of stents 67 described in this application can be covered (within stent 67, on the outside of stent 67 or both) partially or in full with prosthetic graft material (stent-graft) or can be a metal or plastic stent without any prosthetic fabric covering. In stent 67 with a concave flair (FIGS. 14 and 17), the graft going from the larger flared end 71 (FIG. 18) to the smaller end 75 of stent-graft 67 can be attached to or immediately adjacent to convex support struts 70. Or, as shown in a cut-away segment in FIG. 19, the prosthetic material 77-78 attached to larger ring 71 can be attached directly at 79-80 to smaller ring 75 without being adjacent to support struts 70 so that, in this manner, this portion of the flared stent-graft 67 could protect struts 70 from fraying a prosthetic graft inserted into the flared stent-graft 67. In subsequent figures, the designation of stent 67 refers to any or any combination of the above described stents or any other suitable stent.

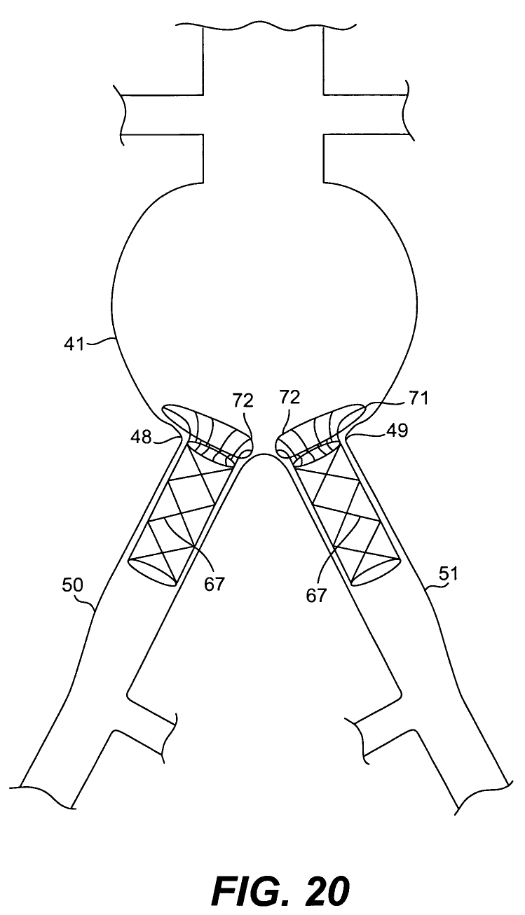

FIG. 20 depicts an abdominal aortic aneurysm 41 with flared 71 stents 67 that are asymmetric 72, in order to avoid one flared 71 stent 67 compressing the stent 67 in the contra-iliac artery. These stents are placed in the right 50 and left 51 iliac arteries with the flared portion 71 extending into the lumen of the aneurysm at the orifice 48 of the right common iliac artery and the orifice 49 of the left common iliac artery.

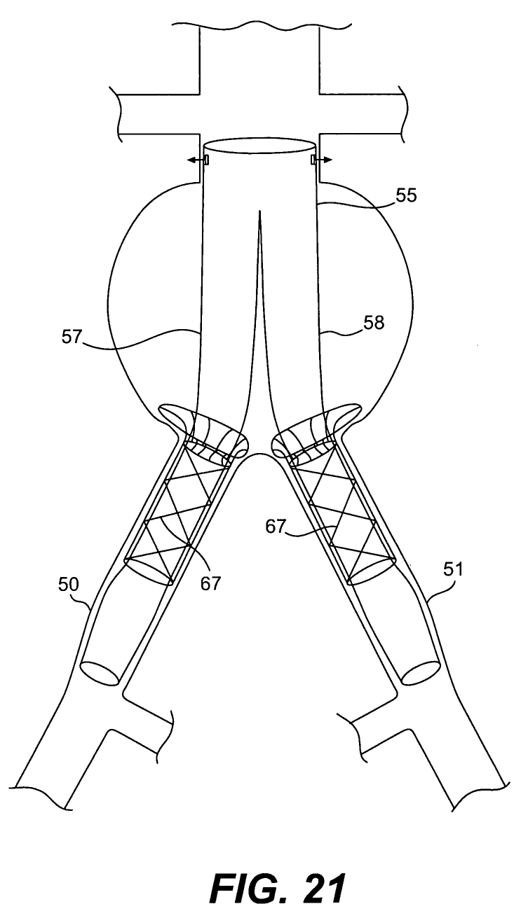

FIG. 21 depicts the right 57 and left 58 limbs of a prosthetic bifurcation graft 55 of one embodiment of the invention passing through previously placed stents 67 in the right 50 and left 51 common iliac arteries. The stents 67 are intended to prevent graft limb complications including kinking, narrowing or occlusion at the orifices or within the common iliac arteries.

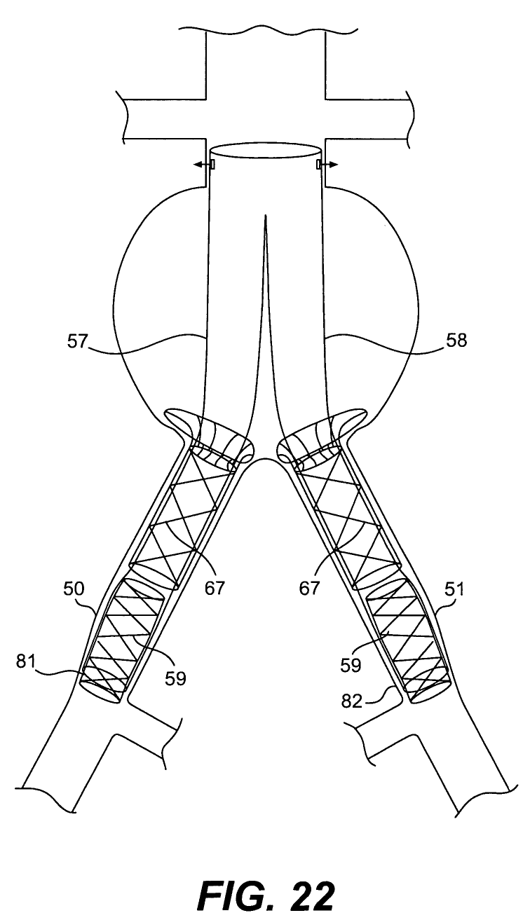

FIGS. 22 through 25 are various methods according to embodiments of the present invention to secure the limbs of the prosthetic graft to the iliac arteries in such a way as to reduce the likelihood of the graft limbs rubbing against a metal strut with each cardiac pulsation and causing fabric deterioration.

FIG. 22 depicts an embodiment of the limbs 57, 58 of the prosthetic graft of the present invention traversing through the lumen of the stents 67 and being attached to the common iliac arteries 50, 51 with stents 59 such that the right 81 and left 82 distal ends of the prosthetic graft are compressed against the inner surface of the common iliac arteries 50, 51. This approach drawn in FIG. 22 may not fully prevent the possibility of fabric wear of the limbs 57, 58 against the stent 67 material but is an acceptable approach.

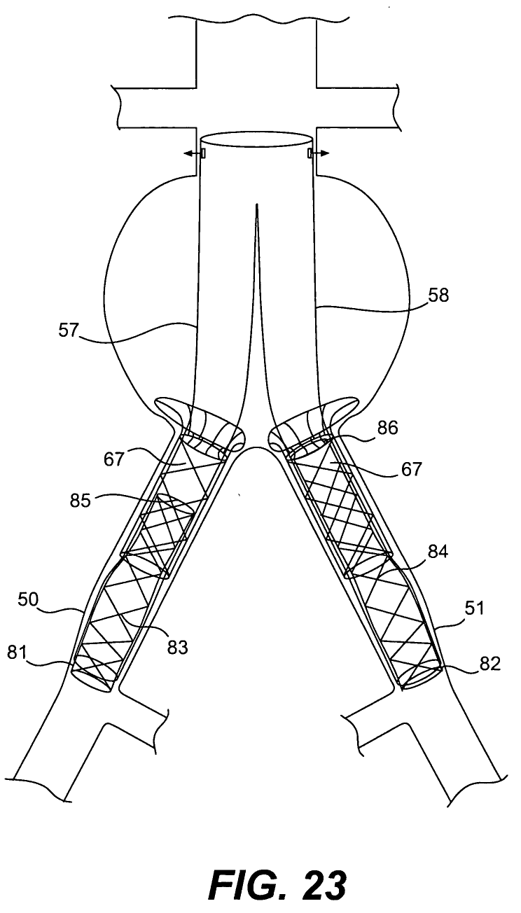

FIG. 23 depicts two additional embodiments of graft limb 57, 58 fixations. On the right, the graft limb 57 traverses the orificial iliac stent or stent-graft 67 and is attached to the iliac artery 50 with a stent or second stent-graft 83 that extends from the end 81 of the graft limb up to the proximal end 85 that is positioned at about the mid portion of the right stent 67. On the left, in another embodiment, the graft limb 58 traverses the orificial iliac stent or stent-graft 67 and is attached to the iliac artery 51 with a stent or stent-graft 84 that extends from the end 82 of the graft limb up to the proximal end 86 that is positioned at about the top portion of the left stent 67.

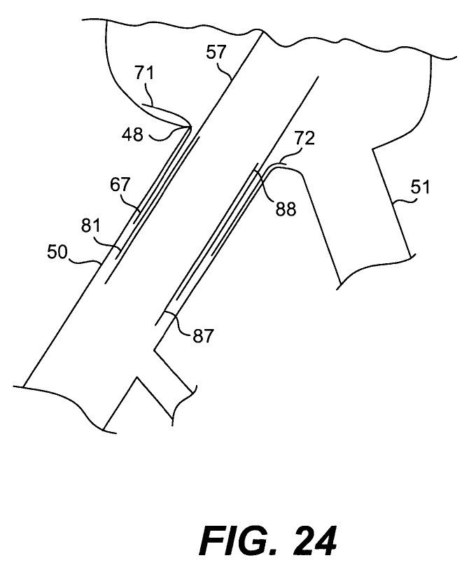

FIG. 24 is a blow-up of a right common iliac artery 50 with an iliac asymmetrical stent (see also FIG. 14) to show the desired relationships among the stents 67, 87; iliac orifice 48; and prosthetic graft 57 of one embodiment of the present invention. The flared portion 71 of the stent 67 is lateral and the asymmetric portion 72 is medial in order to avoid interfering with another stent 67 if it were to be placed within the left common iliac artery 51. The distal end 81 of the right limb 57 of the prosthetic graft traverses the iliac stent 67. Within that graft is placed a stent 87 to compress the distal portion of the graft limb 57 from the orifice of the iliac artery 48 to the end of the graft 81. The proximal end 88 of the stent 87 is at the same level as the orifice of the iliac orifice 48 and at the portion of the stent 67 that begins to flair. In this way, the graft limb may be free of surrounding material until it enters the iliac orifice at which point it may be securely pinioned between the two stents. “Stents” can be simple stents, stents partially covered with prosthetic fabric material, or complete stent-grafts. Additionally the iliac orificial stent 67 and the stent 87 placed within the prosthetic graft can be constructed so there is a tongue and grove or male-female or any other suitable relationship so as to reduce further the likelihood that any of the three components (orificial stent 67, graft limb 57 and inner stent 87) will move independently of any of the other two components.

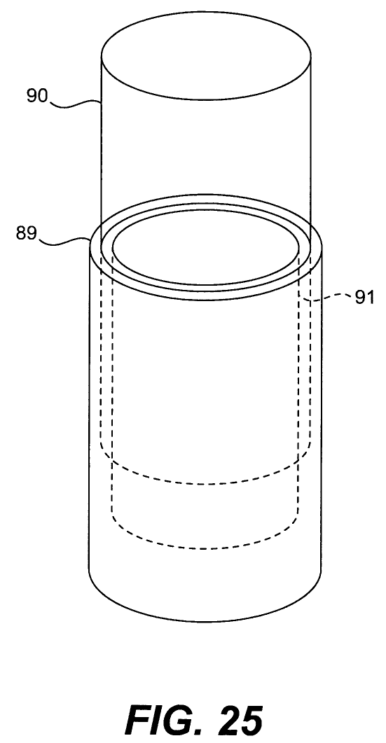

Finally, FIG. 25 illustrates the three-cylinder concept. At the orifice of the common iliac artery, a standard stent or stent-graft 89 is placed with its proximal end right at the orifice of the iliac artery. The graft limb 90 traverses the lumen of this stent 89. An inner stent 91 is placed within the graft limb. This stent 91 can be shorter, longer or the same length as the outer stent 90. Stents 89 and 91 may have incorporated into their design a locking mechanism (tongue and groove or male/female indentations) that enhance the goal of eliminating to as great an extent possible any movement between the graft 90 and either or both stents 89, 91.

Numerous characteristics and advantages have been set forth in the foregoing description, together with details of structure and function. The novel features are pointed out in the appended claims. The disclosure, however, is illustrative only, and changes, may be made in detail, especially in matters of shape, size, and arrangement of parts, within the principle of the invention, to the full extent indicated by the broad general meaning of the terms in which the appended claims are expressed.

1. A method of treating an aneurysm comprising the steps of;
inserting a first flared stent having a lumen into an artery with the flared portion extending into the lumen of the aneurysm,
inserting a bifurcation graft through the lumen of the first flared stent, and
attaching the bifurcation graft to the artery.

2. The method of claim 1 the first flared stent having an asymmetrical flare having a first flared portion and a first unflared portion.

3. The method of claim 1 further including the step of inserting a second flared stent having a lumen into an adjacent artery wherein the bifurcation graft comprise of a first leg and a second leg wherein the first leg passes through the lumen of the first flared stent and the second leg passes through the lumen of the second flared stent.

4. The method of claim 3 the second flared stent having an asymmetrical flare having a second flared portion and a second unflared portion wherein the first flared portion of the first flared stent extends away from the second flared portion of the second flared stent.

5. The method of claim 3 further comprising the step attaching at least one leg of the bifurcation graft to the artery.

6. The method of claim 1 further comprising the steps of;
inserting a repair catheter with an expandable device into the artery with a stricture and
ablating the stricture using the expandable device.

7. The method of claim 6 further comprising the step of using an occlusive device to capture material from the ablated stricture.

8. The method of claim 6 wherein the expandable device ablates the stricture using vibration or ultrasound.

9. The method of claim 1 wherein at least one surgical fastener is used to attach the bifurcation graft to the artery.

10. The method of claim 1 further comprising the step of connecting the bifurcation graft to the flared stent.

11. The method of claim 10 wherein the bifurcation graft and the flared stent are connected by a locking mechanism.

12. The method of claim 1 wherein the aneurysm being repaired is an abdominal aortic aneurysm.

13. A method of treating an aneurysm comprising the steps of;
inserting a first flared stent having a lumen into an artery with the flared portion extending into the lumen of the aneurysm,
inserting a bifurcation graft through the lumen of the first flared stent,
attaching the bifurcation graft to the artery, and
inserting a first inner stent into the lumen of the bifurcation graft.

14. The method of claim 13 the first flared stent having an asymmetrical flare having a first flared portion and a first unflared portion.

15. The method of claim 13 further including the steps of;
inserting a second flared stent having a lumen into an adjacent artery wherein the bifurcation graft further comprises of a first leg and a second leg wherein the first leg passes through the lumen of the first flared stent and the first inner stent is inserted into the lumen of the first leg and
inserting a second inner stent into the lumen of the second leg wherein the second leg passes through the lumen of the second flared stent.

16. The method of claim 15 the second flared stent having an asymmetrical flare having a second flared portion and a second unflared portion wherein the first flared portion of the first flared stent extends away from the second flared portion of the second flared stent.

17. The method of claim 15 further comprising the step attaching at least one leg of the bifurcation graft to the artery.

18. The method of claim 15 further comprising the step of connecting at least one leg of the bifurcation graft to either the first flared stent or the second flared stent.

19. The method of claim 18 wherein the legs of the bifurcation graft and the first and second flared stent are connected by a locking mechanism.

20. The method of claim 15 further comprising the step of connecting at least one leg the bifurcation graft to either the first inner stent or the second inner stent.

21. The method of claim 20 wherein the legs of the bifurcation graft and either the first or second inner stent are connected by a locking mechanism.

22. The method of claim 13 wherein the aneurysm being repaired is an abdominal aortic aneurysm.

23. The method of claim 13 wherein at least one surgical fastener is used to attach the bifurcation graft to the artery.

24. A prosthetic bifurcation graft for repairing an aneurysm comprising,
a main body having a first leg having a first leg lumen defined by a first leg inner wall and a second leg having a second leg lumen defined by a second leg inner wall and
a first flared stent having a first flared stent lumen defined by a first flared stent inner wall wherein the first flared stent inner wall is in communication with the first leg of the main body.

25. The prosthetic bifurcation graft of claim 24 wherein the first flared stent is connected to the first leg of the bifurcation device by a locking mechanism.

26. The prosthetic bifurcation graft of claim 24 further comprising a second flared stent defined by a second flared stent inner wall wherein the second flared stent inner wall is in communication with the second leg of the main body.

27. The prosthetic bifurcation graft of claim 26 wherein the second flared stent is connected to the second leg of the bifurcation device by a locking mechanism.

28. The prosthetic bifurcation graft of claim 26 the first flared stent having an asymmetrical flare having a first flared portion and an first unflared portion and the second flared stent having an asymmetrical flare having a second flared portion and a second unflared portion.

29. The prosthetic bifurcation graft of claim 28 wherein the first flared portion of the first flared stent extends away from the second flared portion of the second flared stent.

30. The prosthetic bifurcation graft of claim 24 further comprising a first inside stent in communication with the first leg inner wall.

31. The prosthetic bifurcation graft of claim 26 further comprising a second inside stent in communication with the second leg inner wall.
